(19) United States
(12) Patent Application Publication
(10) Pub. No.: US 2020/0390917 A1
(43) Pub. Date: Dec. 17, 2020

(54) MOBILE PHONE SANITIZATION DEVICE

(71) Applicant: PROTV DEVELOPMENT INC., Taipei City (TW)

(72) Inventor: Jack TANG, NEW TAIPEI CITY (TW)

(21) Appl. No.: 16/527,493

(22) Filed: Jul. 31, 2019

(30) Foreign Application Priority Data
Jun. 13, 2019 (TW) 108207516

(51) Int. Cl. A61L 2/10

(52) U.S. Cl. CPC A61L 2/10; A61L 2202/16

(57) ABSTRACT

A mobile phone sanitization device using short-wavelength ultraviolet (UVC) requires only one single light source. With the smooth surface of a closed chamber that accommodates the mobile phone and the retention distance between the mobile phone and the closed chamber, short-wavelength ultraviolet is reflected to every surface of the mobile phone. At the same time, the Hall sensor ensures that the ultraviolet lamp is turned off when the cover of the mobile phone sanitization device is opened, preventing ultraviolet light from being irradiated to the human body, achieving both efficient and safe effects.

### BACKGROUND OF THE INVENTION

### 1. Field of the Invention

The present invention relates to mobile phone sanitization technology and more particularly, to a mobile phone sanitization device that uses short-wavelength ultraviolet (UV-C) light to sanitize a mobile phone placed therein.

### 2. Description of the Related Art

In today's society, the mobile phone has clearly become an indispensable tool in people's lives. Riven by advances in technology, the versatility of mobile phones has increased year by year, and at the same time, the frequency of people using mobile phones has also increased. In addition to driving the development of mobile phone-related industries, this phenomenon has also produced a series of derivative problems, and health problems are one of them.

In order to solve the above problem, U.S. Pat. No. 8,964,405 B2 discloses a personal electronic device sanitization device, which comprises a case configured to receive a personal electronic device, a support member configured to maintain the personal electronic device at a particular position and/or orientation, and upper and lower emitters configured to emit electro-optical radiation into the interior portion from the upper portion and the lower region to sanitize the personal electronic device. However, this conventional patent design requires two light sources. The use of electro-optical radiation for sanitization results in high manufacturing costs. Therefore, this design of personal electronic device sanitization device is not conducive to market sales.

### SUMMARY OF THE INVENTION

The present invention has been accomplished under the circumstances in view. It is the main object of the present invention to provide a mobile phone sanitization device whose sanitization method and usage mode can make the user clean the mobile phone more efficiently and safely.

To achieve this and other objects of the present invention, a mobile phone sanitization device comprises a base, a cover, a magnet, an ultraviolet lamp, a button, a power port, a Hall sensor and a startup circuit. The base comprises a downwardly recessed open-top recess, a side recess located on one side wall of the open-top recess and a plurality of convex bumps raised from the bottom of the open-top recess. The open-top recess has a smooth inner surface. The side recess has the opening thereof facing toward the open-top recess. The cover can be opened and closed to the base, having a smooth bottom surface. The cover closes the open-top recess so that the open-top recess forms a closed chamber when the cover is closed to the base. The magnet is mounted in the cover. The ultraviolet lamp is disposed at the base and located at the side recess for illuminating ultraviolet light into the closed chamber. The button is disposed at the base. The power port is disposed on the base for electrically connecting with an external device. The Hall sensor is disposed inside the base for sensing the magnetic force of the magnet. The startup circuit is mounted in the base and electrically connected to the ultraviolet lamp, the button, the power port and the Hall sensor. The open-top recess is configured to accommodate a mobile phone, allowing the accommodated mobile phone to be supported on the convex bumps, so that when the cover is closed to the base, the accommodated mobile phone is kept away from the bottom surface of the cover and the bottom of the open-top recess by a predetermined distance and also kept away from the ultraviolet lamp a retention distance. Through the predetermined distance and the retention distance, the ultraviolet light of the ultraviolet lamp is irradiated to the inner surface of the open-top recess and the bottom surface of the cover and reflected to the outer surface of the mobile phone.

In this way, only one single light source of the ultraviolet light can effectively sanitize the surface of the mobile phone, and at the same time it can ensure that no ultraviolet light will be exposed to the outside of the mobile phone sanitization device when the cover is opened, which is efficient and safe.

Preferably, the cover is pivotally connected to the base by a pivot.

Preferably, the cover comprises an endless vertical wall and an endless wing. The endless vertical wall is inserted into the open-top recess and close proximity to the side wall of the open-top recess and the endless wing is attached to an upper surface of the base when the cover is closed to the base.

Preferably, the magnet is located at one side of said cover.

Preferably, the power port is a USB port.

Preferably, the Hall sensor is disposed inside the base and close to the side wall of the open-top recess.

Preferably, the startup circuit further comprises a boost converter.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is an elevational view of a mobile phone sanitization device in accordance with the present invention.

FIG. 2 corresponds to FIG. 1, showing the cover opened.

FIG. 3 is an exploded view of the mobile phone sanitization device in accordance with the present invention.

FIG. 4 is a sectional view taken along line 4-4 of FIG. 1.

FIG. 5 is a sectional view taken along line 5-5 of FIG. 1.

FIG. 6 is a circuit block diagram of the mobile phone sanitization device in accordance with the present invention.

FIG. 7 is similar to FIG. 4, showing the open-top recess imaginarily accommodated a mobile phone.

FIG. 8 is similar to FIG. 5, showing the open-top recess imaginarily accommodated a mobile phone.

### DETAILED DESCRIPTION OF THE INVENTION

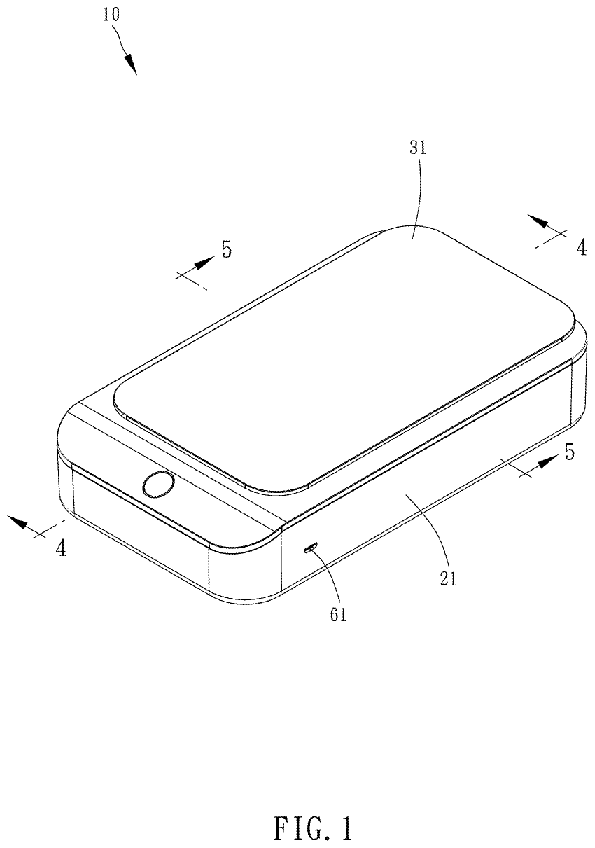

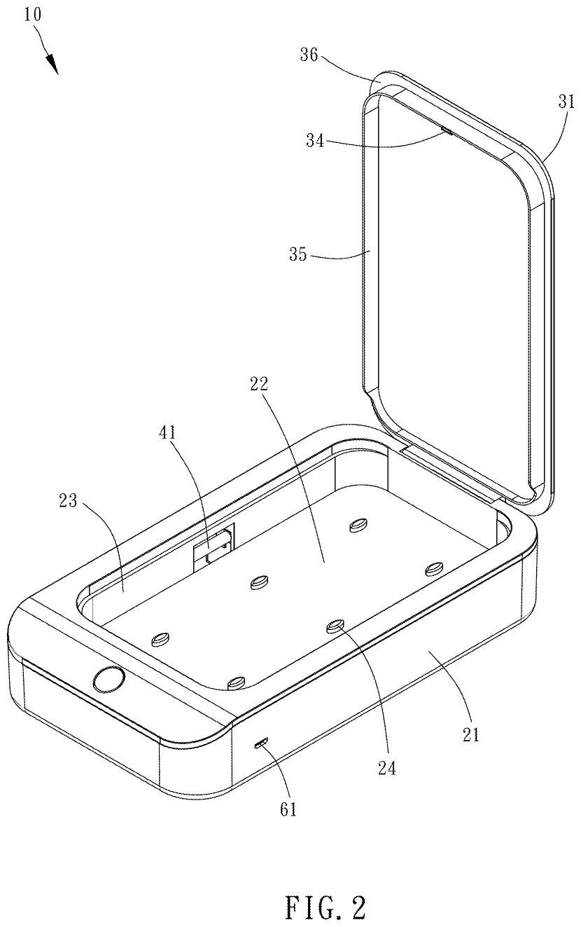

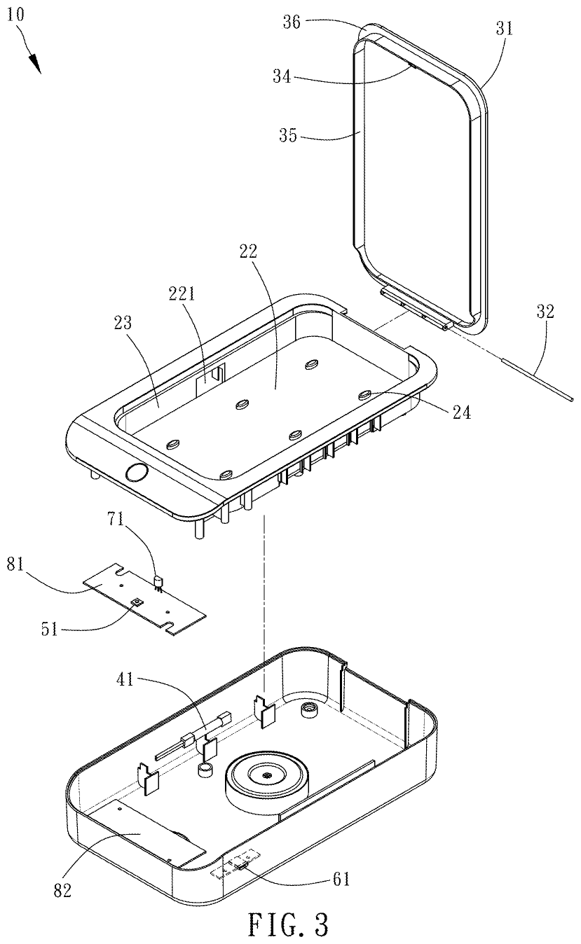

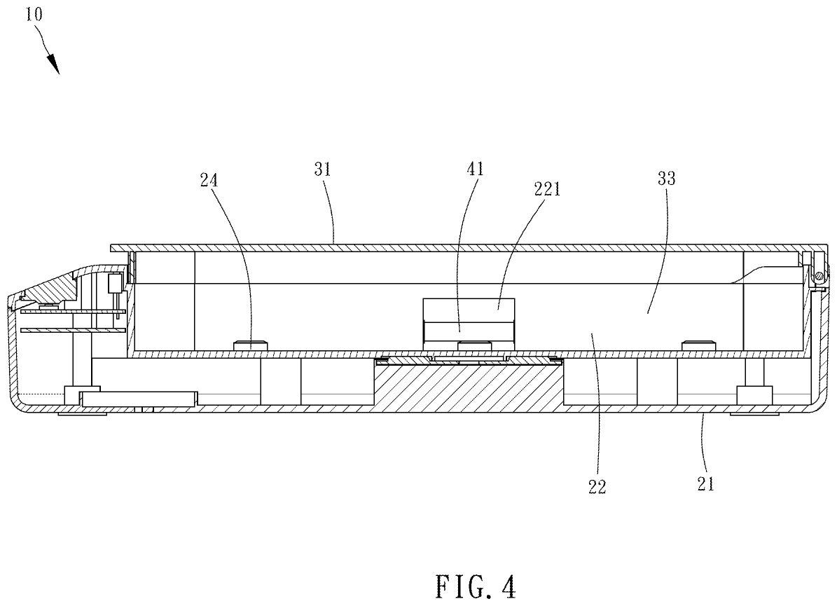

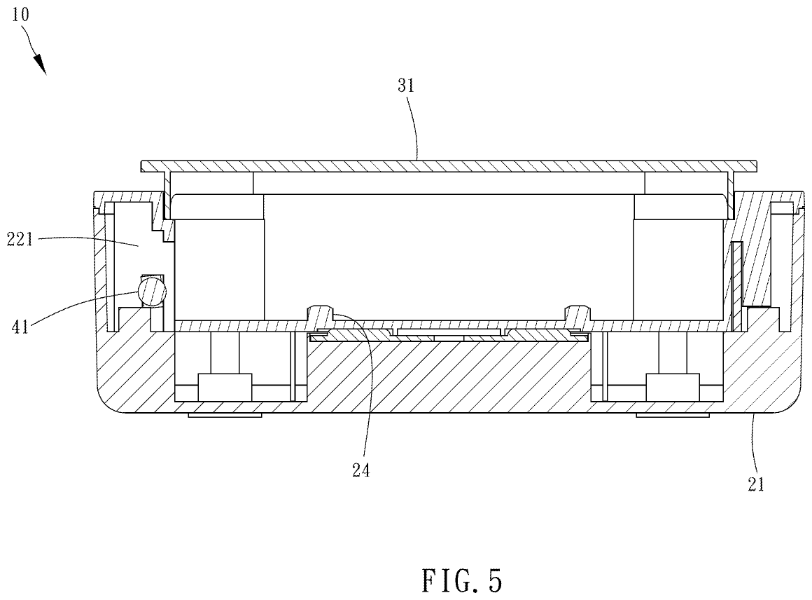

Referring to FIGS. 1-6, a mobile phone sanitization device 10 in accordance with the present invention is shown. The mobile phone sanitization device 10 comprises a base 21, a cover 31, an ultraviolet lamp 41, a button 51, a power port 61, a Hall sensor 71 and a startup circuit 81.

The base 21 has a downwardly recessed open-top recess 22, and a side recess 221 inwardly recessed from one side wall 23 of the open-top recess 22 with the opening thereof facing toward the open-top recess 22. The inner surface of the open-top recess 22 is a smooth surface, and the open-top recess 22 is configured to accommodate a mobile phone 99. The bottom of the open-top recess 22 has a plurality of upwardly convex bumps 24. In the present preferred embodiment, the open-top recess 22 is an example of an elongated open-top recess but is not limited thereto. Similarly, in the present preferred embodiment, the plural convex bump 24 and the base 21 are integrally formed as an example, but not limited thereto.

The cover 31 is pivotally connected to the base 21 by a pivot 32 so that the cover 31 can be opened or closed to the base 21. The bottom surface of the cover 31 is a smooth surface. Further, the cover 31 has an endless vertical wall 35 and endless wing 36. When the cover 31 is closed to the base 21, the endless vertical wall 35 is inserted into the open-top recess 22 and is close proximity to the side wall 23 of the open-top recess 22, and the endless wing 36 is attached to the upper surface of the base 21 to close the open-top recess 22, so that the open-top recess 22 forms a closed chamber 33. The cover 31 has a magnet 34 located at one side thereof. In the present preferred embodiment, the magnet 34 is exemplified by a permanent magnet (not shown) but is not limited thereto.

The ultraviolet lamp 41 is disposed at the base 21 and located at the side recess 221 for illuminating ultraviolet light (not shown) into the closed chamber 33. In the present preferred embodiment, the ultraviolet lamp 41 is exemplified by short-wave ultraviolet (UVC 245 nm) having an irradiation wavelength of 254 nm but is not limited thereto.

The button 51 is disposed at the base 21.

The power port 61 is disposed on the base 21 for electrically connecting with an external device (not shown). In the present preferred embodiment, the power port 61 is based on USB port, but not limited to it.

The Hall sensor 71 is disposed inside the base 21 and close to the side wall 23 of the open-top recess 22 for sensing the magnetic force of the magnet 34 when the cover 31 is closed to the base 21.

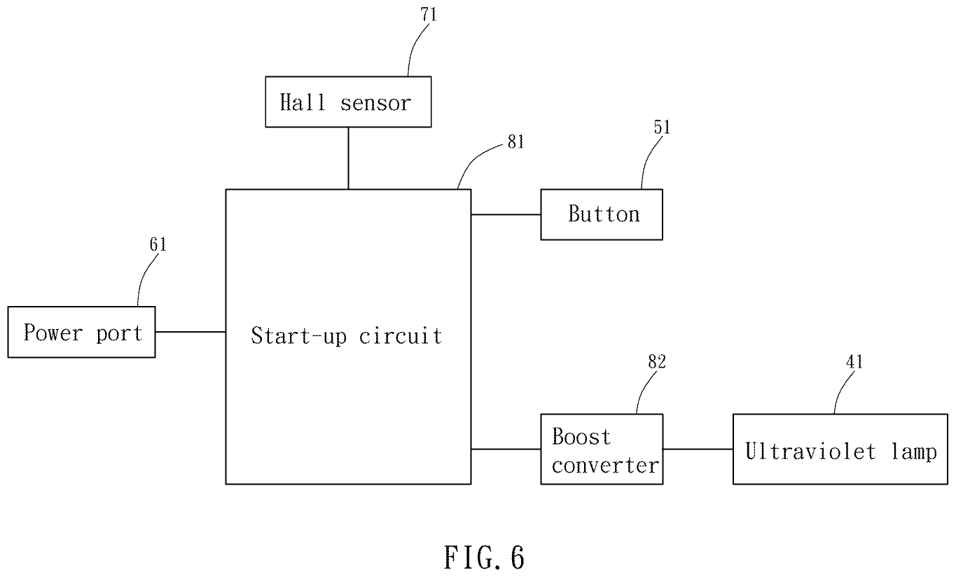

As illustrated in FIG. 6, the startup circuit 81 mounted in the base 21 and electrically connected to the ultraviolet lamp 41, the button 51, the power port 61, the Hall sensor 71 and a boost converter 82. This way of connection is well known and needless to be described here.

The architecture of the preferred embodiment of the present invention has been described above, and the operation state of the preferred embodiment of the present invention will be described next.

First, to use the mobile phone sanitization device 10, a USB cable (not shown) needs to be inserted in the power port 61 to electrically connect the mobile phone sanitization device 10 to an external power source.

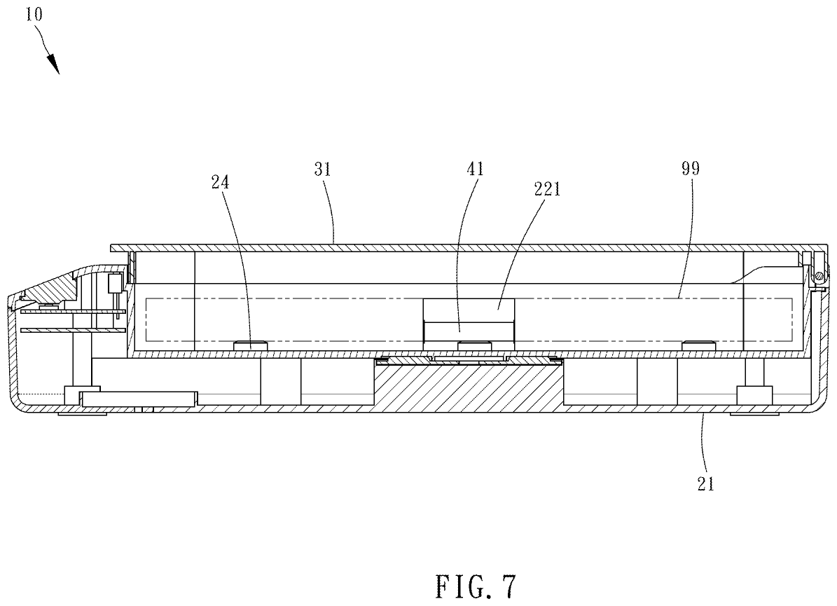

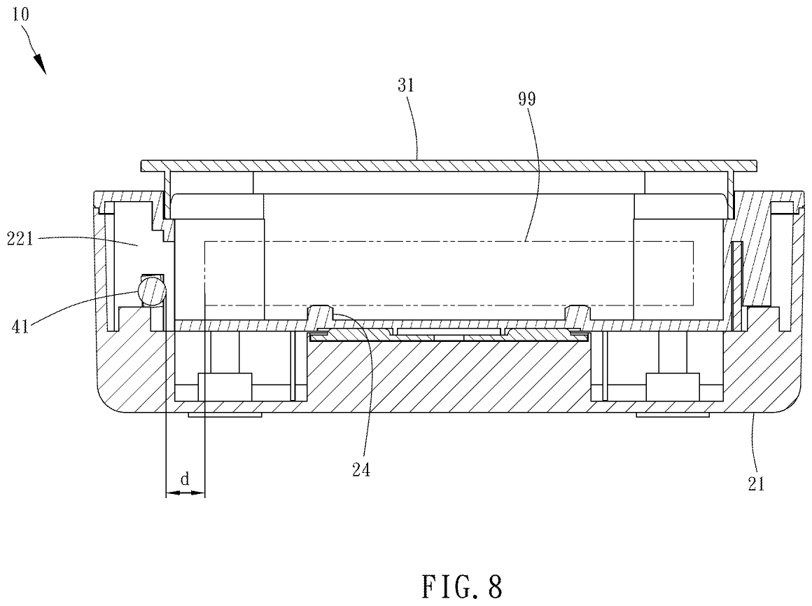

Referring to FIGS. 7 and 8, before the start of the sterilization process, the user opens the cover 31 and places the mobile phone 99 in the open-top recess 22, enabling the mobile phone 99 to be held up by the plural convex bumps 24, so that the mobile phone 99 is kept away from the bottom surface of the open-top recess 22 by a predetermined distance and also kept away from the ultraviolet lamp by a retention distance d.

Then, the user covers the cover 31 to the base 21. When the cover 31 is closed to the base 21, the endless vertical wall 35 is inserted into the open-top recess 22 and is close proximity to the side wall 23 of the open-top recess 22, and the endless wing 36 is attached to the upper surface of the base 21 to close the open-top recess 22, so that the open-top recess 22 forms a closed chamber 33. At this time, the mobile phone 99 is located in the closed chamber 33 and kept away from the bottom surface of the cover 31 by a predetermined distance.

Then, the user presses the button 51 to turn on the startup circuit 81. At this time, the startup circuit 81 receives the potential signal of the Hall sensor 71 as high, indicating that the cover 31 has been actually covered and the Hall sensor 71 has sensed the magnet 34, thus, the startup circuit 81 judges that the sanitization process begins. At the same time, the startup circuit 81 will continue to receive the potential signal of the Hall sensor 71.

After the startup circuit 81 confirms that the cover 31 is actually closed, the startup circuit 81 turns on the ultraviolet lamp 41, causing the ultraviolet lamp 41 to irradiate short-wave ultraviolet rays to the inner surface of the open-top recess 22 and the bottom surface of the cover 31 by the retention distance d. Since the inner surface of the open-top recess 22 and the bottom surface of the cover 31 are smooth surfaces, and the two predetermined distances provide a sufficient angle of reflection, the short-wave ultraviolet rays will be reflected to the outer surface of the mobile phone 99. When short-wave ultraviolet rays are irradiated on the surface of the mobile phone 99, the sanitization effect will be achieved.

In the normal sanitization process, the startup circuit 81 will keep the ultraviolet lamp 41 turning on until the user presses the button 51 again to turn off the startup circuit 81, and the ultraviolet lamp 41 will then be turned off. At this time, the user can open the cover 31, then takes the mobile phone 99 out of the open-top recess 22 to end the sanitization process.

In the case of an abnormal sanitization process, that is, when the cover 31 is opened in the sanitization process, the startup circuit 81 will receive the potential signal of the Hall sensor 71 as low, indicating that the cover 31 is opened. At this time, the startup circuit 81 will turn off the ultraviolet lamp 41 to prevent UVC 245 nm from illuminating the human body, causing damage to the human body.

In addition, for the convenience of use, a timer (not shown) can be additionally electrically connected to the startup circuit 81, so that the startup circuit can determine the term of the sanitization process. When the predetermined time of the sanitization process arrives, the startup circuit 81 will judge that the sanitization process has ended, so the ultraviolet lamp 41 is turned off, and the user can open the cover 31 again and take the mobile phone 99 to complete the sanitization of the mobile phone 99.

What is claimed is:

1. A mobile phone sanitization device, comprising;
a base comprising a downwardly recessed open-top recess, said open-top recess having a smooth inner surface, a side recess located on one side wall of said open-top recess, said side recess having an opening thereof facing toward said open-top recess, and a plurality of convex bumps raised from a bottom of said open-top recess;
a cover openably closed to said base, said cover having a smooth bottom surface and a magnet, said cover closing said open-top recess so that said open-top recess forms a closed chamber when said cover is closed to said base, said magnet mounted in said cover;
an ultraviolet lamp disposed at said base and located at said side recess for illuminating ultraviolet light into said closed chamber;
a button disposed at said base;
a power port disposed on said base for electrically connecting with an external device;
a Hall sensor disposed inside said base for sensing the magnetic force of said magnet;
a startup circuit mounted in said base and electrically connected to said ultraviolet lamp, said button, said power port and said Hall sensor;
wherein said open-top recess is configured to accommodate a mobile phone, allowing the accommodated mobile phone to be supported on said convex bumps, so that when said cover is closed to said base, the accommodated mobile phone is kept away from the bottom surface of said cover and the bottom of said open-top recess by a predetermined distance and also kept away from said ultraviolet lamp a retention distance, and through said predetermined distance and said retention distance, the ultraviolet light of said ultraviolet lamp is irradiated to the inner surface of said open-top recess and the bottom surface of said cover and reflected to an outer surface of said mobile phone.

2. The mobile phone sanitization device as claimed in claim 1, wherein said cover is pivotally connected to said base by a pivot.

3. The mobile phone sanitization device as claimed in claim 1, wherein said cover comprises an endless vertical wall and an endless wing, said endless vertical wall being inserted into said open-top recess and close proximity to said side wall of said open-top recess and said endless wing being attached to an upper surface of said base when said cover is closed to said base.

4. The mobile phone sanitization device as claimed in claim 1, wherein said magnet is located at one side of said cover.

5. The mobile phone sanitization device as claimed in claim 1, wherein said power port is a USB port.

6. The mobile phone sanitization device as claimed in claim 1, wherein said Hall sensor is disposed inside said base and close to said side wall of said open-top recess.

7. The mobile phone sanitization device as claimed in claim 1, wherein said startup circuit further comprises a boost converter.
